(19) United States
(12) Patent Application Publication
(10) Pub. No.: US 2015/0217035 A1
(43) Pub. Date: Aug. 6, 2015

(54) Superelliptical Breast Funnel

(71) Applicants: Ashia M. Pollen, Madison, WI (US); Robert J. Harter, La Crosse, WI (US)

(72) Inventors: Ashia M. Pollen, Madison, WI (US); Robert J. Harter, La Crosse, WI (US)

(21) Appl. No.: 14/685,669

(22) Filed: Apr. 14, 2015

(51) Int. Cl. A61M 1/06

(52) U.S. Cl. CPC A61M 1/06

(57) ABSTRACT

A breast pump system includes a one-size-fits-all funnel for fittingly receiving the breast of a lactating woman. In some examples, the funnel has a superelliptical opening that provides a smooth, naturally occurring vent at the funnel's inlet. The vent places the breast-to-funnel circumferential seal closer to the narrow end of the funnel, rather than at the funnel's wider inlet. The seal being closer to the nipple ensures that the nipple remains centrally aligned within the system's nipple-receiving receptacle, regardless of the size and shape of the breast.

### FIELD OF THE DISCLOSURE

The subject invention generally pertains to human breast milk collection systems and more specifically to a vented breast fitting funnel

### BACKGROUND

The superellipse was the name given by the poet and scientist, Piet Hein, for a distinctive elliptical shape defined by a certain formula. The shape of a superellipse appears to be a blend of a circle, an ellipse and a square, but it is not a rounded square. One of the most notable applications of a superellipse was in a proposal that Hein submitted in response to a challenge from the city of Stockholm, Sweden for the design of an efficient roundabout for their city square. In his proposal, Hein explained his design as follows:

“Man is the animal that draws lines which he himself then stumbles over. In the whole pattern of civilization there have been two tendencies, one toward straight lines and rectangular patterns and one toward circular lines. There are reasons, mechanical and psychological, for both tendencies. Things made with straight lines fit well together and save space. And we can move easily—physically or mentally—around things made with round lines. But we are in a straitjacket, having to accept one or the other, when often some intermediate form would be better. To draw something freehand—such as the patchwork traffic circle they tried in Stockholm—will not do. It isn't fixed, isn't definite like a circle or square. You don't know what it is. It isn't esthetically satisfying. The super-ellipse solved the problem. It is neither round nor rectangular, but in between. Yet it is fixed, it is definite it has a unity.”

The shape of superellipses and roundabouts may be unrelated to the shape of funnels found in conventional breast milk collection devices used for collecting breast milk from a lactating woman. Such funnels, or breast guides, have a round inlet opening for fittingly receiving the woman's breast. In many cases, a vacuum pump provides cyclical periods of positive and negative pressure to the milk collection device. During periods of negative pressure (subatmospheric pressure), vacuum delivered to the device withdraws a small discrete volume of milk from the breast and conveys that charge of milk to a small charging chamber. During each period of positive pressure, lightly pressurized air relaxes the breast momentarily while at the same time forces the charge of milk from the charging chamber to a larger milk storage chamber. The cycle repeats until the storage chamber is full or until the woman is finished “pumping.”

The funnel, or breast guide, of some breast pump systems are worn within the cup of a common brassiere. Examples of such systems are disclosed in U.S. Pat. Nos. 7,559,915; 8,118,772; and 8,702,646; all of which are incorporated herein by reference. Other breast pump systems have funnels that are handheld or are supported by or extend through a special purpose brassier. Examples of such systems are disclosed in U.S. Pat. Nos. 5,941,847; 7,094,217; and 8,057,452; all of which are incorporated herein by reference.

### BRIEF DESCRIPTION OF THE DRAWINGS

FIG. 1 is a cross-sectional side view of an example milk collection device constructed in accordance with the teachings disclosed herein.

FIG. 2 is a combination schematic diagram and cross-sectional side view similar to FIG. 1 but showing the milk collection device as part of an example breast pump system.

FIG. 3 is a view similar to FIG. 2 but showing the system during a positive pressure period rather than a suction pressure period.

FIG. 4 is a cross-sectional side view of the milk collection device shown in FIGS. 1-3, but showing the device fully tipped over and pointed down.

FIG. 5 is a cross-sectional view of the milk collection device shown in FIG. 1 but showing the device in a disassembled cleaning state.

FIG. 6 is a cross-sectional view similar to FIG. 1 but with the outer shell omitted.

FIG. 7 is a cross-sectional view showing a portion of FIG. 6.

FIG. 8 is a cross-sectional view taken along line 8-8 of FIG. 7.

FIG. 9 is a cross-sectional view showing a portion of FIG. 6.

FIG. 10 is a cross-sectional view taken along line 10-10 of FIG. 9.

FIG. 11 is a cross-sectional view showing a portion of FIG. 6.

FIG. 12 is a cross-sectional view taken along line 12-12 of FIG. 11.

FIG. 13 is a cross-sectional view showing a portion of FIG. 6.

FIG. 14 is a cross-sectional view taken along line 14-14 of FIG. 13.

FIG. 15 is a cross-sectional view similar to FIG.10 but showing an airflow pattern during a negative pressure period (first period).

FIG. 16 is a cross-sectional view similar to FIG. 15 but showing an airflow pattern during a positive pressure period (second period).

FIGS. 17 and 18 are illustrations demonstrating an example “vacuum break” concept.

FIG. 19 is an illustration demonstrating another example “vacuum break” concept.

FIG. 20 is a cross-sectional view similar to FIG. 1 but showing another example milk collection device constructed in accordance with the teachings disclosed herein.

FIG. 21 is a cross-sectional view similar to FIG. 1 but showing another example milk collection device constructed in accordance with the teachings disclosed herein.

FIG. 22 is a cross-sectional view similar to FIG. 1 but showing of another example milk collection device constructed in accordance with the teachings disclosed herein.

FIG. 23 is a cross-sectional side view of an example breast pump system constructed in accordance with the teachings disclosed herein, wherein a breast is about to engage the system's breast guide.

FIG. 24 is a cross-sectional side view similar to FIG. 23 but showing initial contact between the breast and the breast guide.

FIG. 25 is a cross-sectional side view similar to FIG. 24 but showing the breast in deeper contact with the breast guide.

FIG. 26 is a cross-sectional side view similar to FIG. 25 but showing vacuum drawing the breast even further into the breast guide.

FIG. 27 is a cross-sectional side view showing a milk collection system similar to the system shown in FIGS. 23-26 but without ventilating means for ensuring proper alignment of a nipple within a nipple receptacle.

FIG. 28 is a cross-sectional side view similar to FIG. 28 but showing the nipple misaligned with the nipple receptacle.

FIG. 29 is a cross-sectional side view of an example breast guide and nipple receptacle usable in the milk collection device shown in FIGS. 23-26.

FIG. 30 is a cross-sectional view taken along line 30-30 of FIG. 29.

FIG. 31 is a cross-sectional view taken along line 31-31 of FIG. 29.

FIG. 31A is a cross-sectional view similar to FIG. 31 but showing a breast within the breast guide.

FIG. 32 is a cross-sectional view taken along line 32-32 of FIG. 29.

FIG. 33 is a cross-sectional side view of an example breast guide and nipple receptacle usable in the milk collection device shown in FIGS. 23-26.

FIG. 34 is a cross-sectional view taken along line 34-34 of FIG. 33.

FIG. 35 is a cross-sectional view taken along line 35-35 of FIG. 33.

FIG. 35A is a cross-sectional view similar to FIG. 35 but showing a breast within the breast guide.

FIG. 36 is a cross-sectional view taken along line 36-36 of FIG. 33.

FIG. 37 is a cross-sectional side view of an example breast guide and nipple receptacle usable in the milk collection device shown in FIGS. 23-26.

FIG. 38 is a cross-sectional view taken along line 38-38 of FIG. 37.

FIG. 39 is a cross-sectional view taken along line 39-39 of FIG. 37.

FIG. 39A is a cross-sectional view similar to FIG. 39 but showing a breast within the breast guide.

FIG. 40 is a cross-sectional view taken along line 40-40 of FIG. 37.

FIG. 41 is a cross-sectional side view of an example breast guide and nipple receptacle usable in the milk collection device shown in FIGS. 23-26.

FIG. 42 is a cross-sectional view taken along line 42-42 of FIG. 41.

FIG. 43 is a cross-sectional view taken along line 43-43 of FIG. 41.

FIG. 44 is a cross-sectional view taken along line 44-44 of FIG. 41.

FIG. 45 is a cross-sectional side view of an example breast guide and nipple receptacle usable in the milk collection device shown in FIGS. 23-26.

FIG. 46 is a cross-sectional view taken along line 46-46 of FIG. 45.

FIG. 47 is a cross-sectional view taken along line 47-47 of FIG. 45.

FIG. 48 is a cross-sectional view taken along line 48-48 of FIG. 45.

FIG. 49 is a cross-sectional side view of an example breast guide and nipple receptacle usable in the milk collection device shown in FIGS. 23-26.

FIG. 50 is a cross-sectional view taken along line 50-50 of FIG. 49.

FIG. 51 is a cross-sectional view taken along line 51-51 of FIG. 49.

FIG. 52 is a cross-sectional view taken along line 52-52 of FIG. 49.

### DETAILED DESCRIPTION

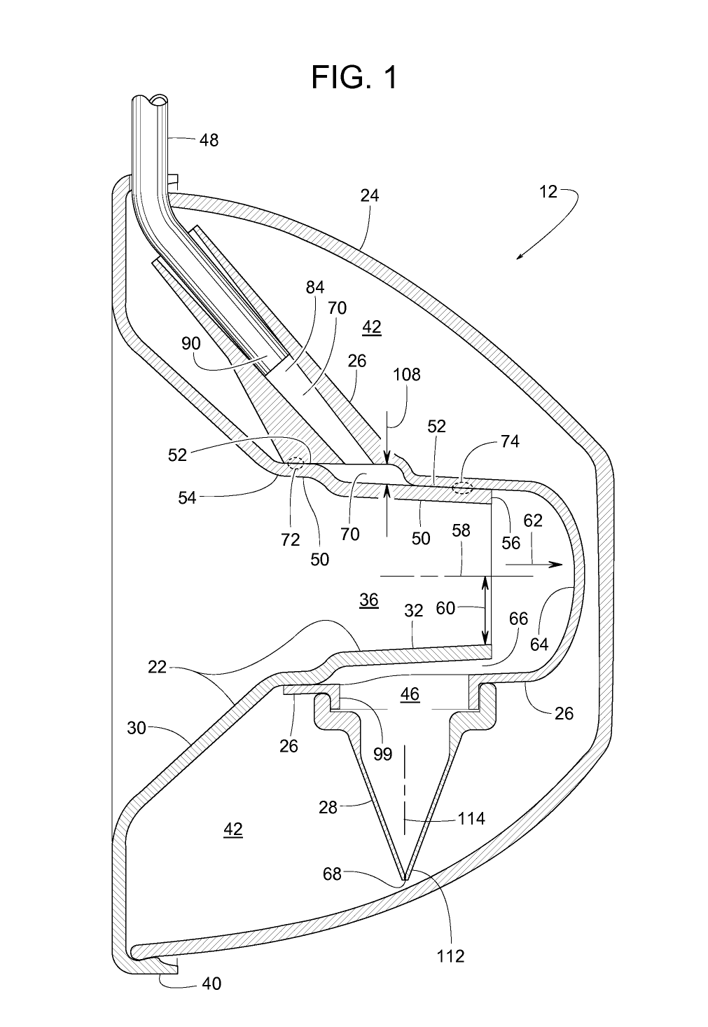

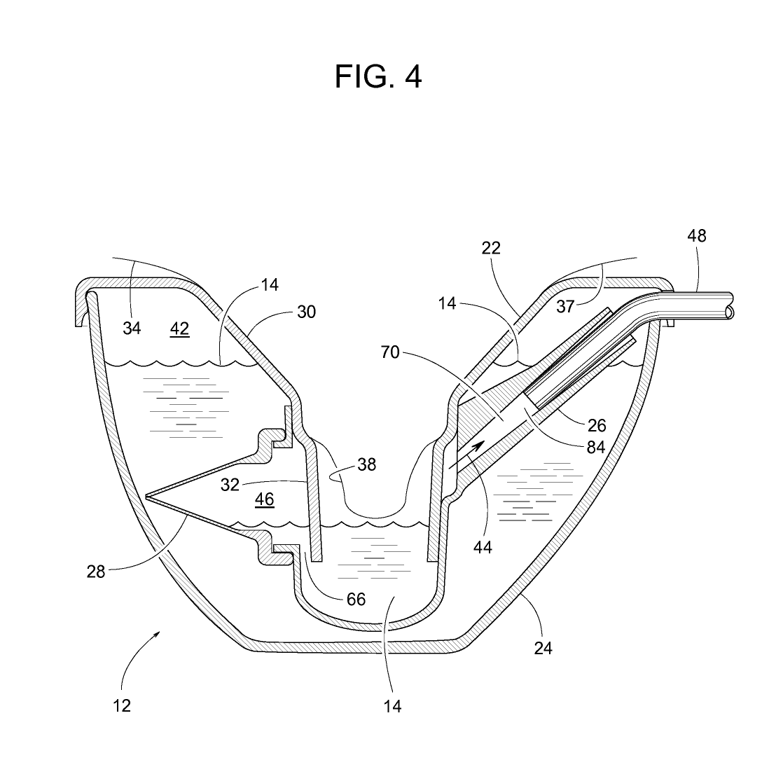

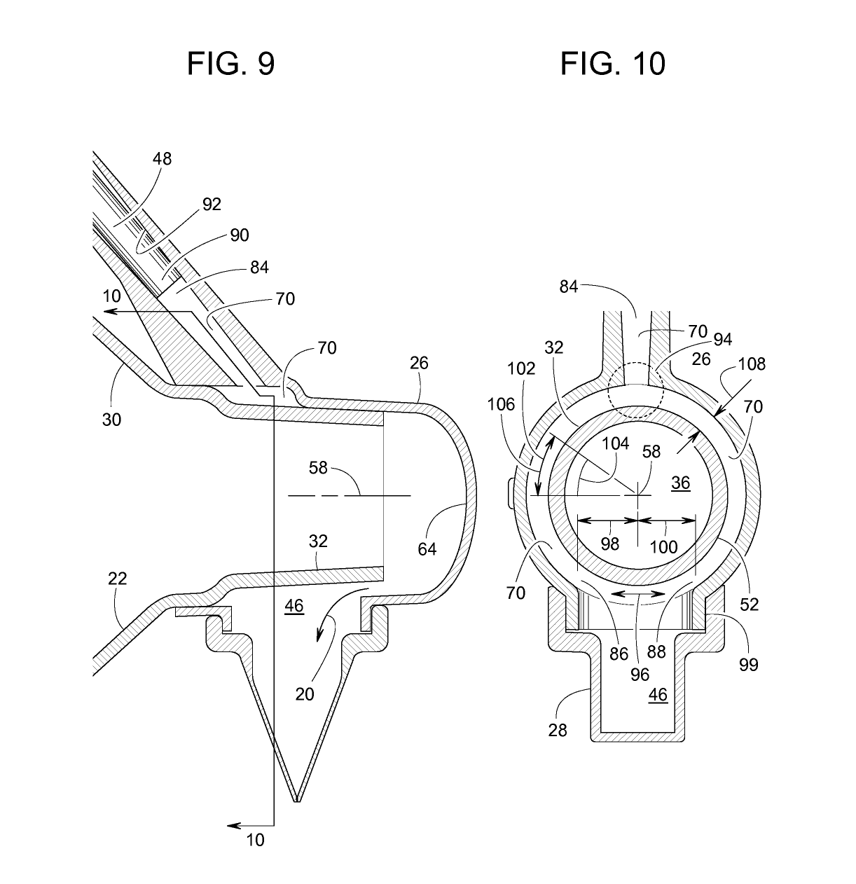

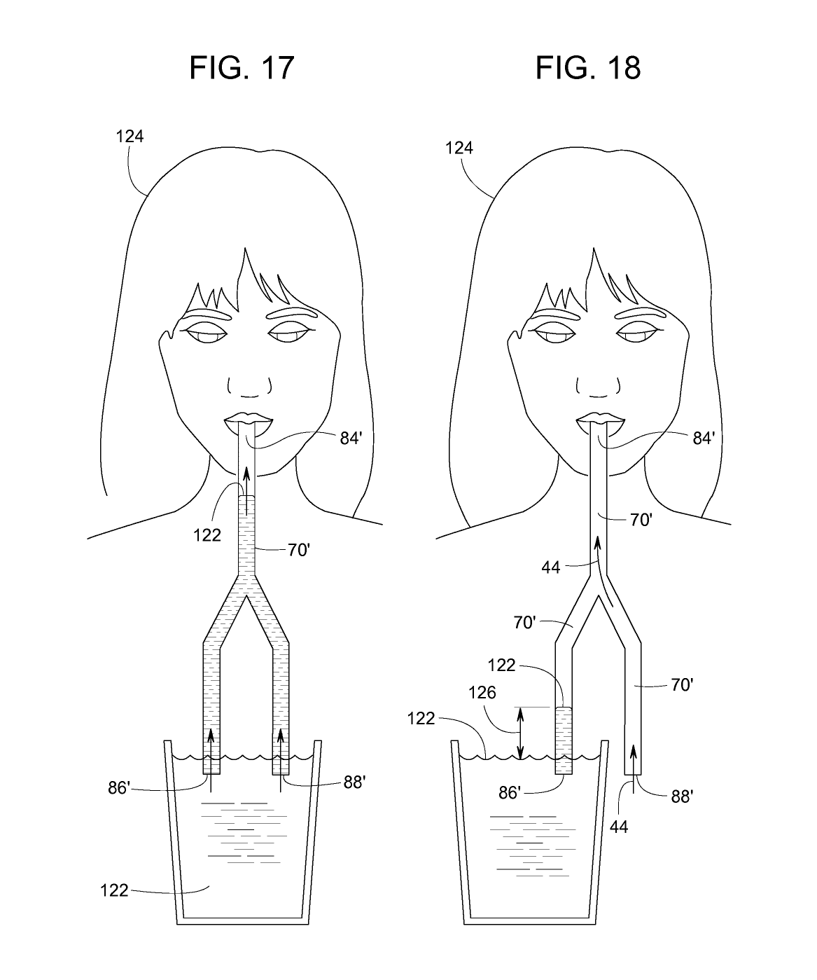

FIGS. 1-16 show various views of an example breast pump system 10 that includes a milk collection device 12 with means for preventing milk 14 from backflowing to a vacuum pump 16. FIGS. 17-19 illustrate the underlying operating principle of vacuum breakers. And FIGS. 21-22 show variations of the system design. The general design isolates a subatmospheric air flow path 102 (FIG. 10) from a milk flow path 20 (FIG. 9) even if milk collection device 12 it tipped completely over (FIG. 4). The vacuum breaker concept keeps fluids separated without using conventional baffles, which inherently have crevices that can be difficult to clean.

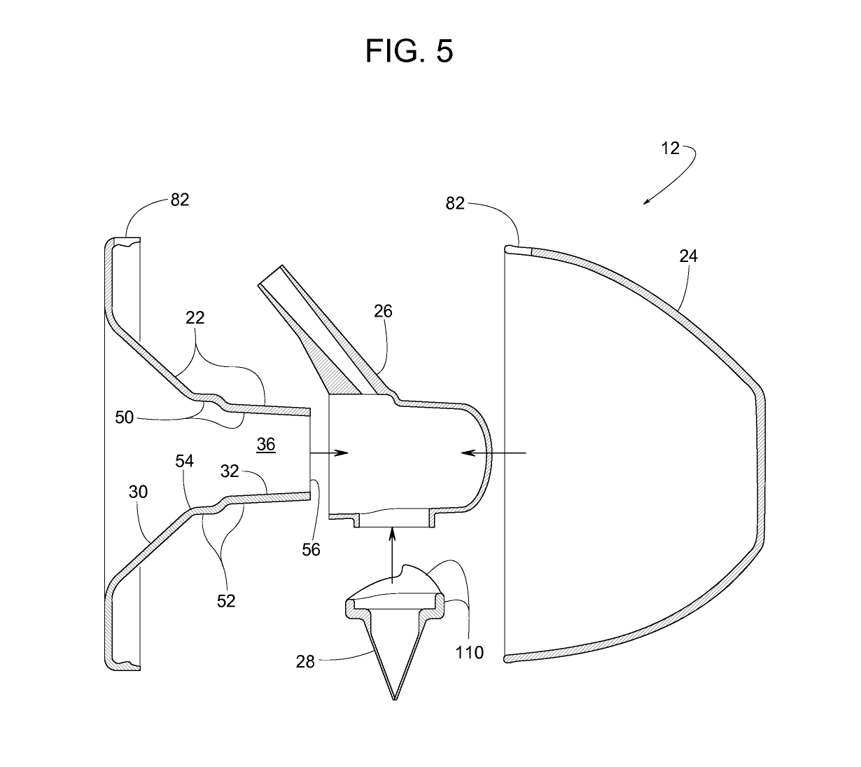

As an overview of the breast pump system's general construction, milk collection device 12 comprises four main parts: a funnel-shaped breast receiver 22, a domed outer shell 24, a fluid exchanger 26, and a unidirectional valve 28 (e.g., a check valve, a duckbill check valve, a reed valve, a ball check valve, a diaphragm check valve, a swing check valve, etc.). FIG. 1 shows these for main parts in an assembled operating state with the parts being positioned as a unit in a predetermined orientation, and FIG. 5 shows them in a disassembled cleaning state. Breast receiver 22 itself comprises a breast guide 30 and a nipple receptacle 32. Breast guide 30 is generally conical for fittingly receiving a breast 34 of a lactating woman 36, and nipple receptacle 32 is tubular and defines a nipple chamber 36 for receiving a nipple 38 of breast 34.

In some examples, outer shell 24 removably connects to a flange 40 of breast receiver 22 to define a milk storage chamber 42 between outer shell 24 and breast receiver 22. Fluid exchanger 26 is coupled to breast receiver 22 to provide means for strategically directing milk 14 and air 44 within milk collection device 12. Valve 28 establishes a milk charging chamber 46 between nipple receptacle 36 and storage chamber 42. In some examples, charging chamber 46 is cycled between positive and negative pressure to draw discrete quantities of expressed milk from nipple receptacle 36. During periods of positive pressure, charging chamber 46 discharges each discrete quantity or charge through valve 28 to storage chamber 42.

To provide charging chamber 46 with air 44 cyclically at subatmospheric pressure and positive or atmospheric pressure, a suction tube 48 couples milk collection device 12 to vacuum pump 16. The term, “vacuum pump,” refers to any device that provides subatmospheric pressure continuously, cyclically, or at least momentarily. Vacuum pump 16 is schematically illustrated to represent all types of vacuum pumps, examples of which include, but are not limited to, a diaphragm pump, a bellows pump, a piston pump, a reciprocating pump, a peristaltic pump, a positive displacement pump, a gear pump, a lobed rotor pump, a screw compressor, a scroll compressor, and a rotary vane pump.

The breast pump system's structure and operation can be further understood with additional definitions and explanations of some detailed features of the system. Nipple receptacle 36 has an inner curved wall surface 50, an outer curved wall surface 52, a proximate end 54 and a distal end 56. The nipple receptacle's tubular shape defines a longitudinal centerline 58 and nipple chamber 30. A minimum radial distance 60 exists between longitudinal centerline 58 and inner curved wall surface 50, wherein the minimum radial distance is measured perpendicular to centerline 58. Nipple receptacle 36 extends longitudinally in a forward direction 62 (parallel to centerline 58) from proximate end 54 to distal end 56. In some examples, nipple chamber 36 extends farther forward than distal end 56 of nipple receptacle 32; however, any part of nipple receptacle 32 that happens to extend farther forward than nipple chamber 36 is considered an extension beyond distal end 56 and thus is not considered the receptacle's distal end 56 itself. In some examples, the most forward point of nipple chamber 36 is at a domed concave surface 64 on fluid exchanger 26. Surface 64 being domed rather than flat makes fluid exchanger 26 easier to clean after fluid exchanger 26 is separated from breast receiver 22.

When breast receiver 22 and valve 28 are attached to fluid exchanger 26, the resulting assembly produces various fluid passages, chambers and sealing interfaces. Upon disassembly, the passages, chambers and sealing interfaces become more open for easier cleaning and sanitizing. Examples of such passages, chambers and sealing interfaces include charging chamber 46, nipple chamber 36, a milk passage 66 for conveying milk 14 from nipple chamber 36 to charging chamber 46, a valve outlet 68 that periodically discharges discrete volumes of milk 14 to storage chamber 42, an air duct 70 that connects suction tube 48 in fluid communication with charging chamber 46, a primary sealing interface 72, and a secondary sealing interface 74.

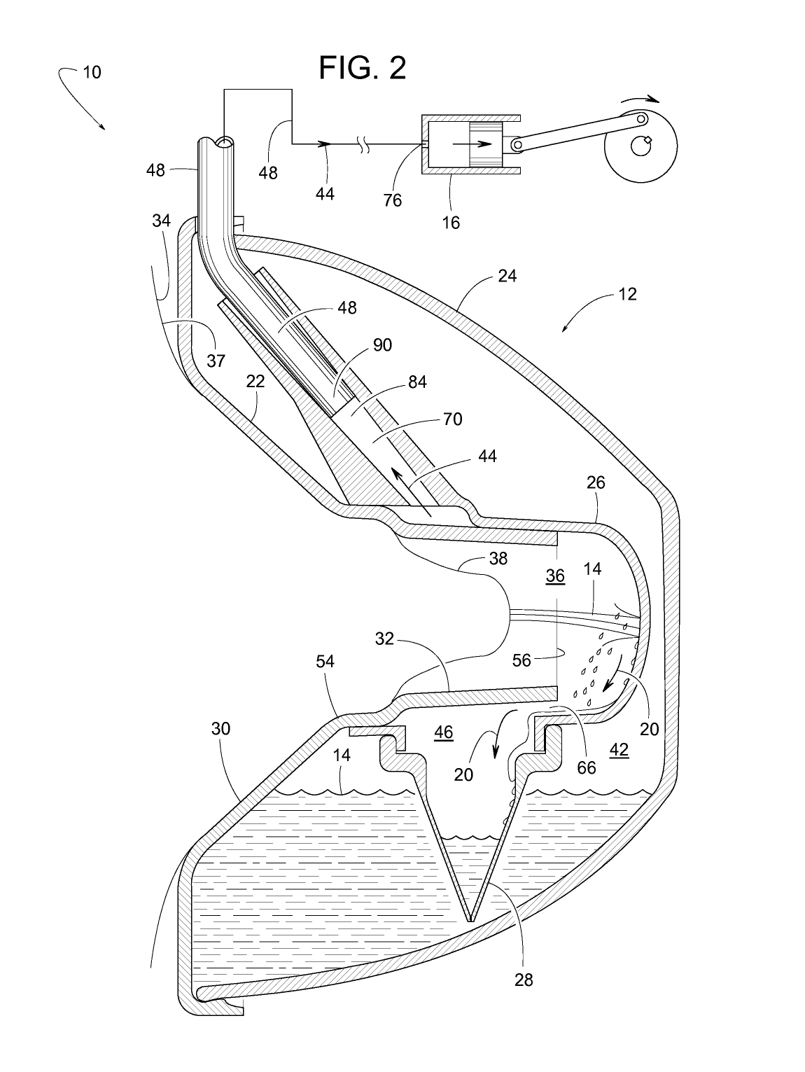

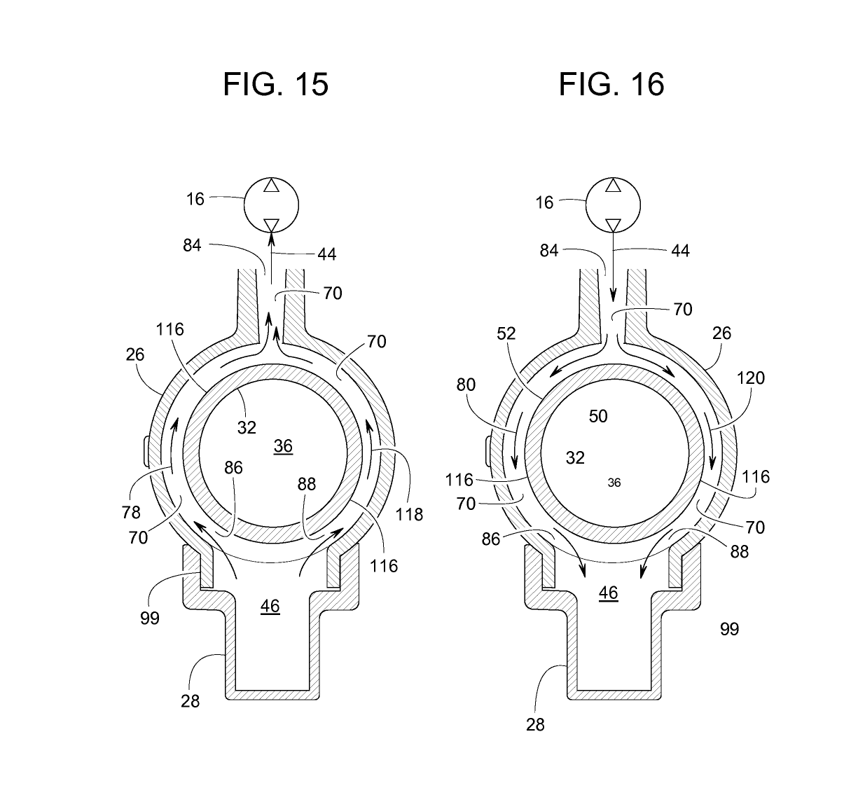

In some examples, system 10 operates in an alternating manner of suction periods and pressurized periods. During suction periods, as shown in FIGS. 2 and 15, vacuum pump 16 applies suction or air at subatmospheric pressure to a remote end 76 of suction tube 48. At least some of the vacuum reaches nipple chamber 36 to draw milk expressed from nipple 38. The expressed milk 14 flows from nipple chamber 36, flows through milk passage 66, and collects at the bottom of charging chamber 46. The negative air pressure produced by vacuum pump 16 creates a first current of air 78 (FIG. 15) that effectively moves from nipple chamber 36 and effectively flows in series through milk passage 66, through charging chamber 46, through air duct 70 (FIGS. 9, 10, 15 and 16), through suction tube 48, and to vacuum pump 16. The terms, “effectively moves” and “effectively flows” means that there is some air movement from an upstream point toward a downstream point, but the air at the upstream point will not necessarily reach the downstream point, due to the travel distance and/or other flow constraints.

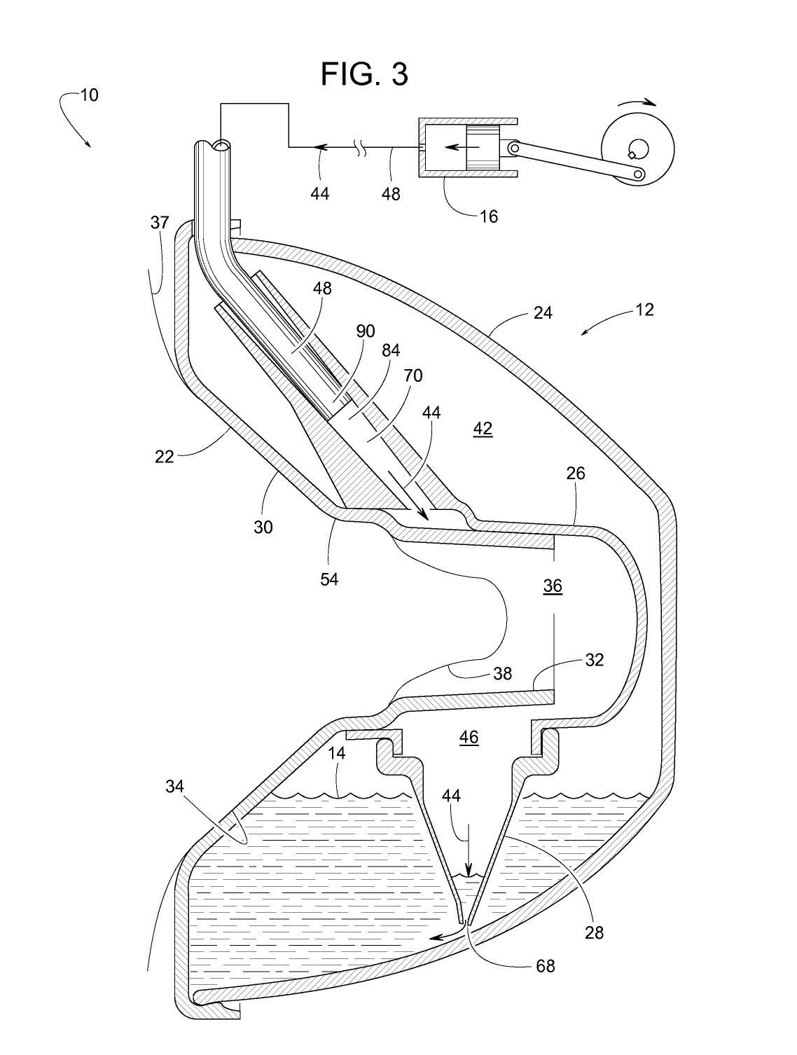

During pressurized periods, as shown in FIGS. 3 and 16, vacuum pump 16 applies positive air pressure to suction tube 48. The positive pressure creates a second current of air 80 that effectively flows in series through suction tube 48, through air duct 70, through milk passage 66, and into nipple chamber 36. The air pressure in charging chamber 46 forces milk 14 (collected during the previous suction period) from charging chamber 46, down through valve 28, and into storage chamber 42. The air pressure in nipple chamber 36 allows breast 34 to relax prior to the next suction period.

The alternating cycle of suction and pressure is repeated for as long as desired or until storage chamber 42 is filled to some predetermined capacity. Upon completion of the pumping process, any suitable means can be used for transferring collected milk from storage chamber 42 to a bottle or to some other convenient storage container. One example method for transferring milk 14 from storage chamber 42 is to pull suction tube 48 out from within an opening 82 (FIG. 5) between breast receiver 22 and outer shell 24, and then pour collected milk 14 out through opening 82. Another method is to turn milk collection device 12 over (e.g., FIG. 4), remove breast receiver 22 from outer shell 24, and simply pour milk 14 out from shell 24.

Although FIG. 4 is referred to illustrate means for emptying milk 14 collected in storage chamber 42, the primary purpose of FIG. 4 is to show how well device 12 tolerates a completely tipped-over condition while still preventing milk 14 from backflowing into suction tube 48. Device 12 has three features that prevent milk backflow. One, in the tipped-over position, air duct 70 remains elevated above milk passage 66. Two, a circumferential seal 74 (FIG. 12) exists between air duct 70 and milk 14 in nipple chamber 36. Three, air duct 70 connects to charging chamber 46 at two spaced apart openings 86 and 88 (see FIG. 15 and the explanation referencing FIGS. 17, 18 and 19)

Preventing milk 14 from entering suction tube 48 is important for several reasons. Milk droplets or even a milk film trapped inside a narrow suction tube can be very difficult to thoroughly clean and sanitize. If left unclean, the trapped milk can contaminate future milk collections. Also, if milk in suction tube 48 migrates into vacuum pump 16, the milk can be even more difficult to remove and can possibly damage or destroy pump 16. Tolerating such unsanitized conditions is generally unheard of in the fields of medicine and food processing.

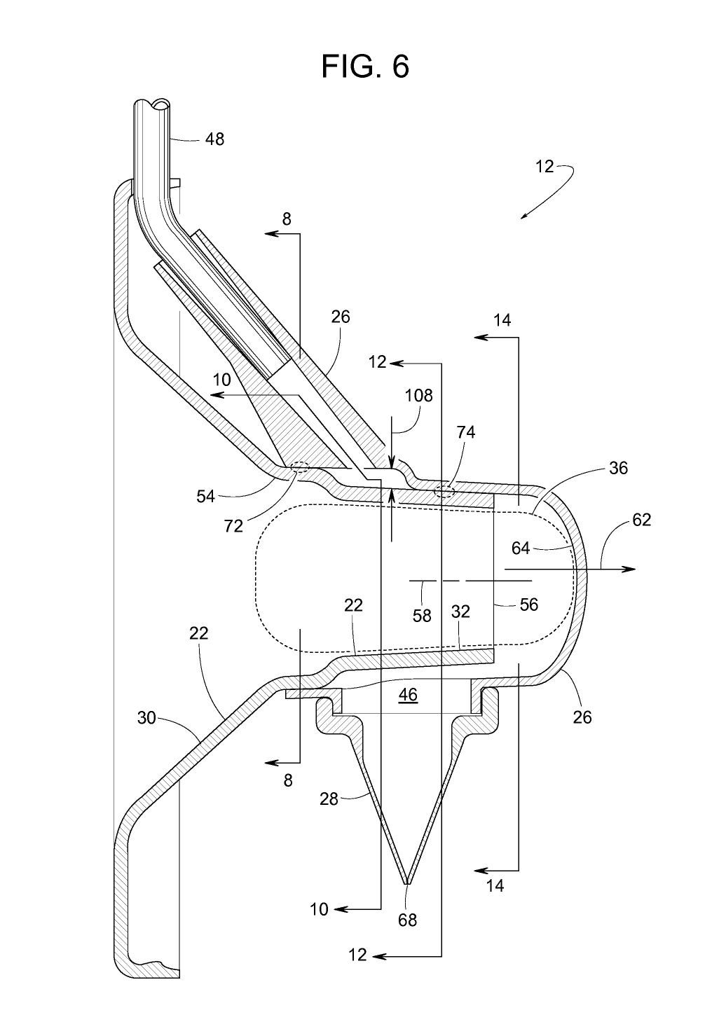

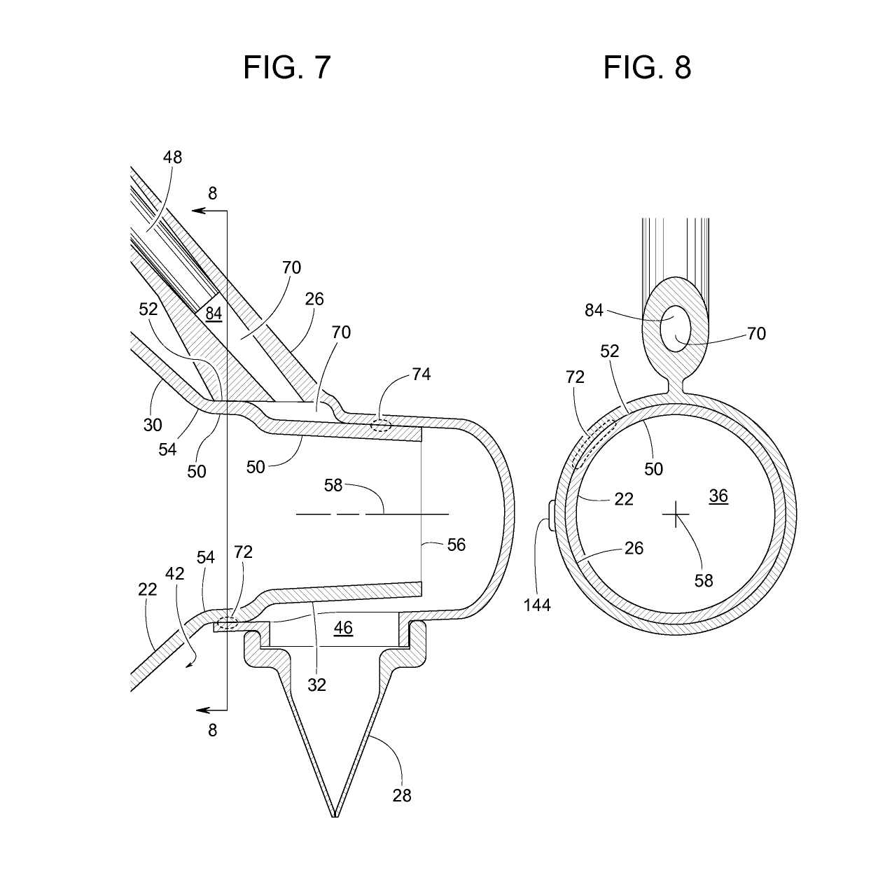

FIG. 6 serves as somewhat of an index drawing for a subsequent series of cross-sectional views. The views in the series are shown in sets of two and are identified as FIGS. 7-8, FIGS. 9-10, FIGS. 11-12, and FIGS. 13-14. FIGS. 7-8 show primary sealing interface 72 between an outer diameter of breast receiver 22 and an inner diameter of fluid exchanger 26. Primary sealing interface 72 is a relatively tight seal that extends 360 degrees circumferentially around centerline 58 to isolate localized pressure or vacuum within charging chamber 46 while the surrounding storage chamber 42 is at atmospheric pressure. In some examples, to ensure a positive seal, interface 72 tapers at 3-degrees in a lengthwise direction with reference to centerline 58.

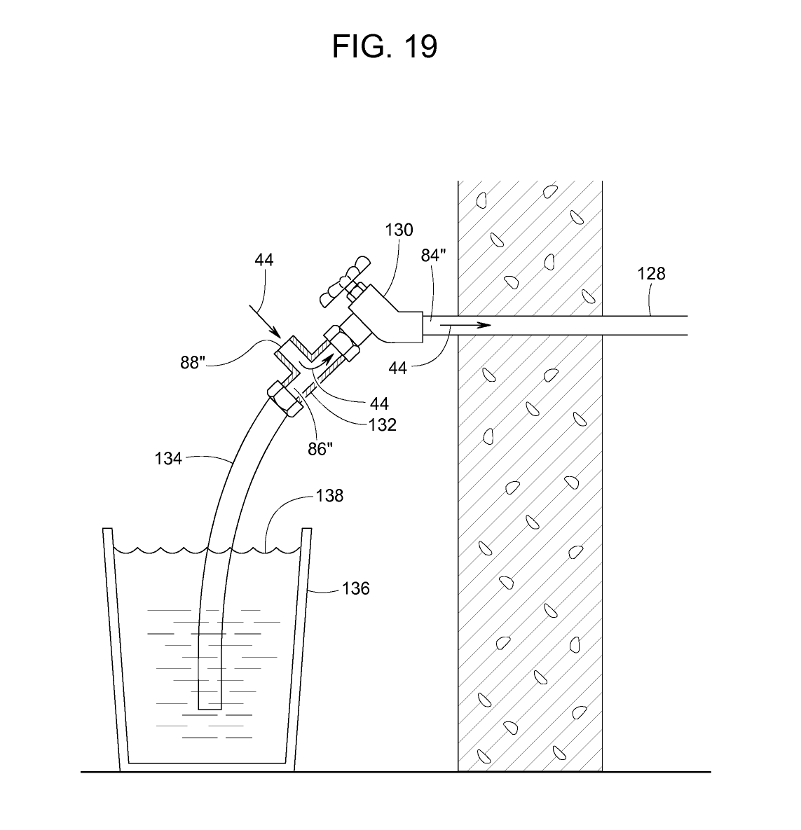

FIGS. 9-10 show one example of air duct 70 connecting vacuum tube 48 in fluid communication with charging chamber 46. In this example, air duct 70 comprises a supply port 84 at a connection end 90 of suction tube 48, a first opening 86 at charging chamber 46, and a second opening 88 at charging chamber 46. To connect tube 48 to supply port 84, connection end 90 of suction tube 48 press-fits into a tapered bore 92 of fluid exchanger 26. A fork 94 (e.g., one path leading to two) in air duct 70 connects supply port 84 in fluid communication with openings 86 and 88. Features 84, 86 and 88 of FIG. 10 correspond respectively to points 84′, 86′ and 88′ of FIG. 18. Features 84, 86 and 88 of FIG. 10 also correspond respectively to points 84″, 86″ and 88″ of FIG. 19.

To apply the “vacuum break” concept illustrated in FIGS. 17 and 18, fork 94 straddles nipple receptacle 36 so that openings 86 and 88 are spaced apart in a lateral direction 96 with the nipple receptacle longitudinal centerline 58 being laterally interposed between openings 86 and 88 (dimensions 98 and 100). In some examples, nipple receptacle 36 is flanked by openings 86 and 88, which means that the nipple's longitudinal centerline 58 is laterally between openings 86 and 88, as shown in FIG. 10. The spaced-apart distance and elevation of openings 86 and 88 can be increased by increasing the diameter of a flange 99 to which valve 28 is attached.

Still referring to FIG. 10, some examples of air duct 70 define a flow path 102 from supply port 84 to first opening 86, wherein a curved section of flow path 102 extends circumferentially an angular distance 104 of at least thirty degrees to avoid having to create an alternate flow path in front of or through nipple chamber 36. In some examples, at least one section 106 of flow path 102 lies within a radial gap 108 between fluid exchanger 26 and the nipple receptacle's outer curved wall surface 52. Upon disassembling device 12 to its disassembled cleaning state (FIG. 5), section 106 of flow path 102 is split apart, which makes flow path 102 and air duct 70 much more accessible for cleaning.

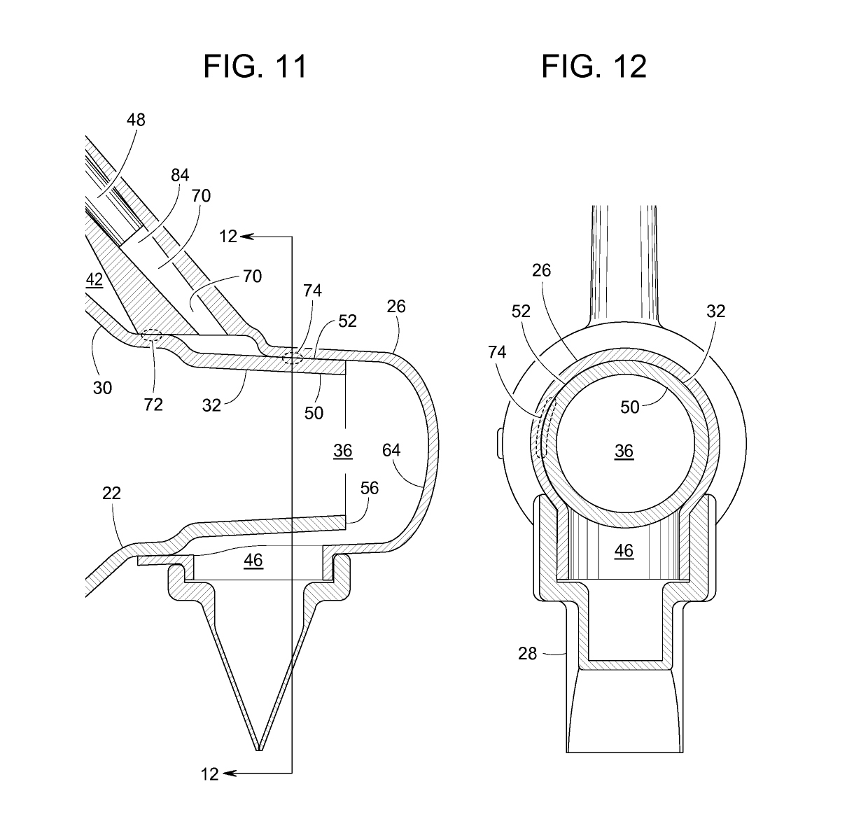

FIGS. 11 and 12 show secondary sealing interface 74 radially between fluid exchanger 26 and the nipple receptacle's outer curved wall surface 52. Secondary sealing interface 74 provides a barrier that prevents milk 14 from flowing directly from nipple chamber 36 to air duct 70. FIG. 11 shows air duct 70 being between primary sealing interface 72 and secondary sealing interface 74.

Primary sealing interface 72 is the more critical seal of the two because primary sealing interface 72 is subjected to an appreciable pressure differential between supply port 84 and storage chamber 42. Secondary sealing interface 74, however, is not as critical because the pressure differential between supply port 84 and nipple chamber 36 is nearly zero. Consequently, in some examples, primary sealing interface 72 is made to be a tighter seal than secondary sealing interface 74. In other words, when breast receiver 22 is snugly inserted into fluid exchanger 26, the radial forces at primary sealing interface 72 is greater than that at secondary sealing interface 74.

It can be important to have primary sealing interface 72 be the dominant seal because when breast receiver 22 is inserted into fluid exchanger 26, something has to “bottom out” first to stop the relative insertion movement of breast receiver 22 into fluid exchanger 26. If secondary sealing surface 74 or distal end 56 abutting domed surface 64 were to be the first parts to bottom out, that might leave some radial clearance or leak path at primary sealing interface 72. Intentionally making primary sealing interface 72 be the first to bottom out, loosens the manufacturing tolerances at other near bottom-out locations, thus increasing assembly reliability, reducing tooling costs, and simplifying manufacturing.

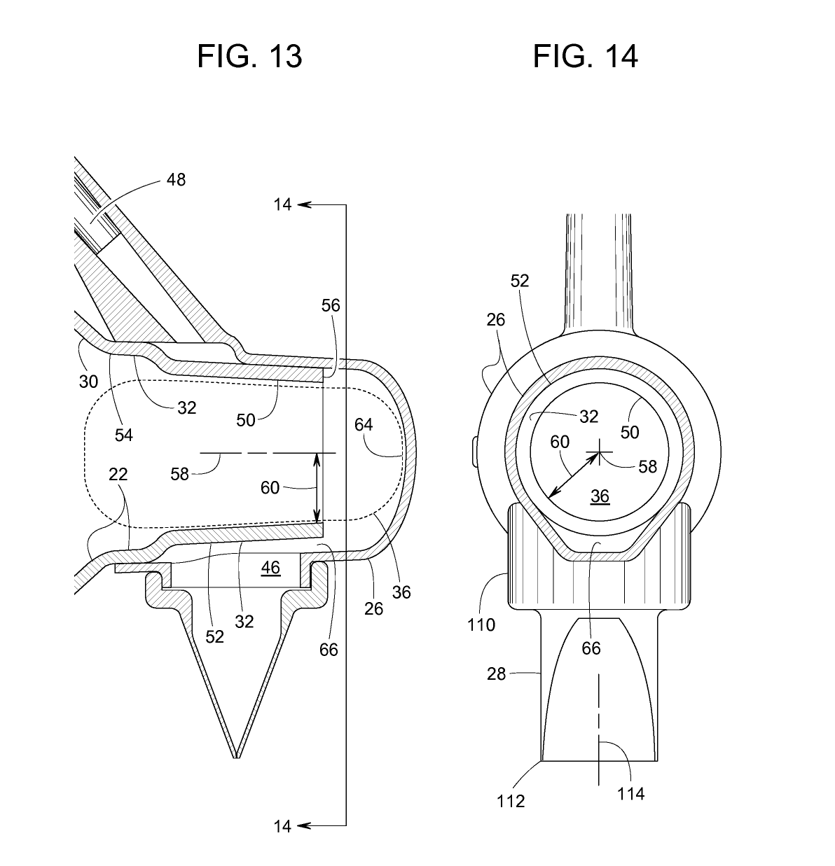

FIGS. 13 and 14 show milk passage 66 between charging chamber 46 and nipple chamber 36. FIGS. 14 and 5 show how an irregular shaped upper flange 110 of valve 28 serves as a means for “clocking” or rotationally aligning valve 28 to fluid exchanger 26. Such alignment can be important to avoid interference between a lower end 112 of valve 28 and outer shell 24. For instance, if valve 28 were rotated ninety degrees (about a vertical axis 114) from the position shown in FIG. 1, the valve's lower end 112 might press up against outer shell 24, whereby outer shell 24 might hold valve 28 open and prevent it from closing.

FIGS. 15 and 16 illustrate an example breast pump method operating during a first suction period (FIGS. 2 and 15) and a second pressure period (FIGS. 3 and 16). FIG. 15 shows during the first period, directing first current of air 78 in a first curved upward direction circumferentially across a first outer convex wall surface 116 of nipple receptacle 32. FIG. 15 also shows during the first period, directing a third current of air 118 in a second curved upward direction circumferentially across the nipple receptacle's first outer convex wall surface 116. FIG. 16 shows during the second period, directing second current of air 80 in a first curved downward direction circumferentially across the nipple receptacle's first outer curved wall surface 116. FIG. 16 also shows during the second period, directing a fourth current of air 120 in a second curved downward direction circumferentially across the nipple receptacle's first outer curved wall surface 116, wherein nipple receptacle 32 is interposed between first current of air 78 and third current of air 118 during the first period, and nipple receptacle 32 is interposed between second current of air 80 and fourth current of air 120 during the second period.

FIGS. 17 and 18 illustrates the concept of a vacuum breaker as a means for preventing a liquid 122 from backflowing up to a suction source 124. Liquid 122 only reaches suction source 124 when both openings 86′ and 88′ are submerged in liquid 122, as shown in FIG. 17. If only one opening 86′ is submerged and the other opening 88′ is exposed to air 44, as shown in FIG. 18, air 44 readily supplies the volume drawn in by suction source 124. Through a given opening, air can flow about thirty times easier than water. Consequently, only a slight pressure differential is needed for air 44 to rush through opening 88′ to suction source 124. That slight pressure differential creates only a slight pressure head 126 that is unable to lift liquid 122 from opening 86′ to suction source 124.

FIG. 19 provides another example of illustrating a vacuum breaker concept. This example involves the use of a residential water line 128, an outdoor faucet 130, a simplified vacuum breaker 132, and a garden hose 134 partially submerged in a bucket 136 of contaminated water 138. In this example, if unusual adverse conditions create a vacuum in water line 128, clean outdoor air 44 rather than contaminated water 138 will be drawn into water line 128.

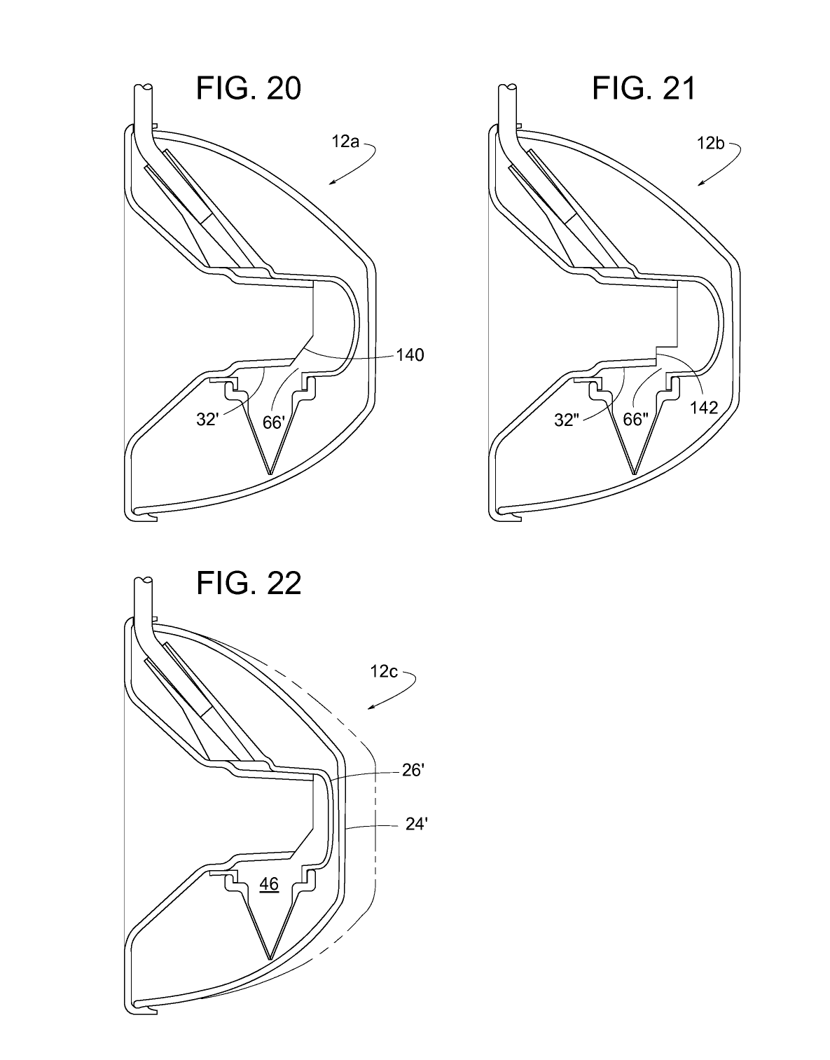

FIGS. 20, 21 and 22 show various design modifications. FIG. 20 shows an altered milk passage 66′ created by a beveled edge 140 at the end of a nipple receptacle 32′. FIG. 21 shows an altered milk passage 66″ created by a notched edge 142 at the end of a nipple receptacle 32″. FIG. 22 shows that a stubbier fluid exchanger 26′ and a less protruding outer shell 24′ can be used when air duct 4 curves around the sides of the nipple receptacle rather than in front of it. The stubbier fluid exchanger 26′ also reduces the effective volume of charging chamber 46, which can be beneficial when using certain low displacement vacuum pumps.

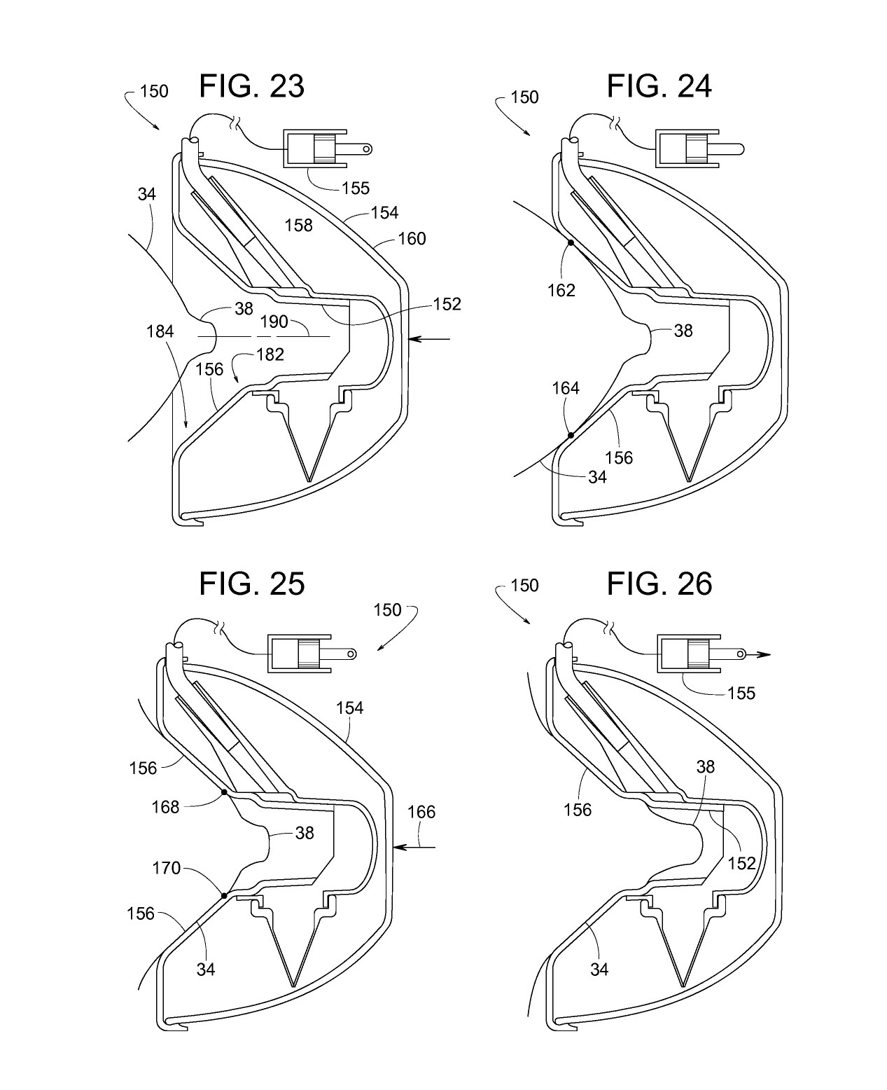

In addition or alternatively, FIGS. 23-26 show an example breast pump system 150 with means for ensuring that nipple 38 is properly positioned within a nipple receptacle 152. In this example, breast pump system 150 comprises a milk collection device 154 and a vacuum pump 155. Milk collection device 154 comprises a funnel-shaped breast guide 156, nipple receptacle 152, a fluid exchanger 158, and an outer shell 160. Breast pump system 150, milk collection device 154, breast guide 156, nipple receptacle 152, fluid exchanger 158, outer shell 160 and vacuum pump 155, shown in FIGS. 23-26, respectively correspond to breast pump system 10, milk collection device 12, breast guide 30, nipple receptacle 32, fluid exchanger 26, outer shell 24 and vacuum pump 16, shown in FIGS. 1-16.

FIG. 23 shows milk collection device 154 being installed onto breast 34. FIG. 24 shows initial light contact between breast 34 and breast guide 156 at points 162 and 164. Upon subsequently applying additional force 166 to device 154, as shown in FIG. 25, contact between breast 34 and breast guide 156 moves deeper into breast guide 156, as indicated by points 168 and 170. FIG. 26 shows vacuum pump 155 applying vacuum that draws nipple 38 into the proper position within nipple receptacle 152. With nipple 38 properly positioned, breast pump system 150 can be operated in a normal manner as described with reference to breast pump system 10.

If breast pump system 150 were to lack means for ensuring proper positioning of nipple 38 within nipple receptacle 152, nipple 38 could become misaligned within the nipple chamber, as shown in FIGS. 27 and 28. For example, if sealing contact were to occur at points 172 and 174, as shown in FIG. 27, subsequent vacuum could draw breast 34 into the milk collection device. However, if more contact pressure or friction exists at point 172 than at point 174, then the area of breast 34 at point 172 could be held stationary while sliding 176 occurs at point 174. Such localized sliding would shift nipple 38 upward, as indicated by arrows 178 and 180. In some cases, as shown in FIG. 28, nipple 38 could become so terribly misaligned and obstructed that breast 34 fails to express milk.

This problem becomes more evident when one considers the suction force of the vacuum as applied across the wide or narrow end of a funnel shaped breast guide. In some cases, the vacuum is −2 psig, the diameter of the wide end is about 70 mm, and the diameter of the narrow end is about 26 mm. In such an example, vacuum sealed at the narrow end could draw a breast in with a reasonable 1.6 lbs of force. Vacuum sealed at the wide end, however, could draw a breast in with about 12 lbs of force, which is greater than the weight of some bowling balls and certainly enough to pull a breast off center.

Such a problem can be more likely to occur if the shape or size of a breast guide does not perfectly match the shape or size of breast 34. It can be impractical and expensive for manufacturers to provide custom shaped breast guides to fit breasts of various sizes. Even a given breast can change in size and shape. To overcome this problem, various examples of breast guide 156 (e.g., breast guides 156a-f) are more adapted to fitting breasts of various shapes and sizes. Breast guide 156 provides a universal fit by initially establishing a seal at a narrow end 182 of breast guide 156, near nipple receptacle 152, while at the same time venting a wide end 184 of breast guide 156.

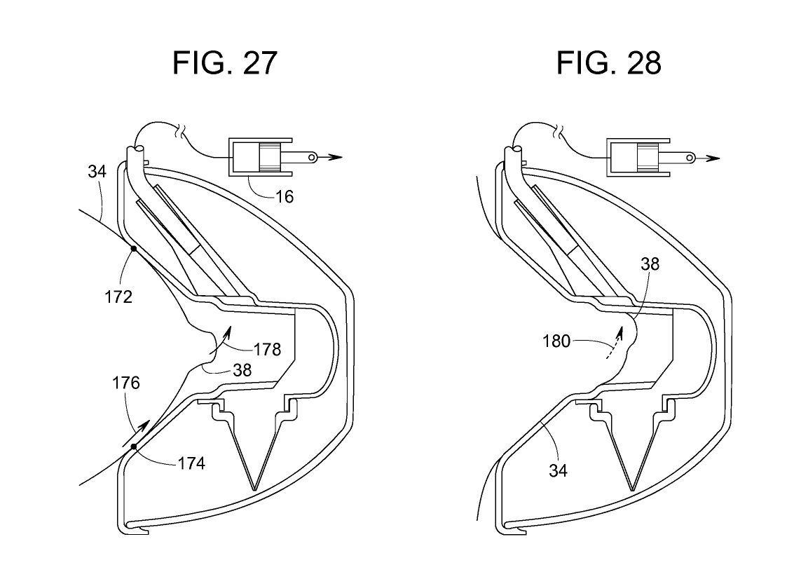

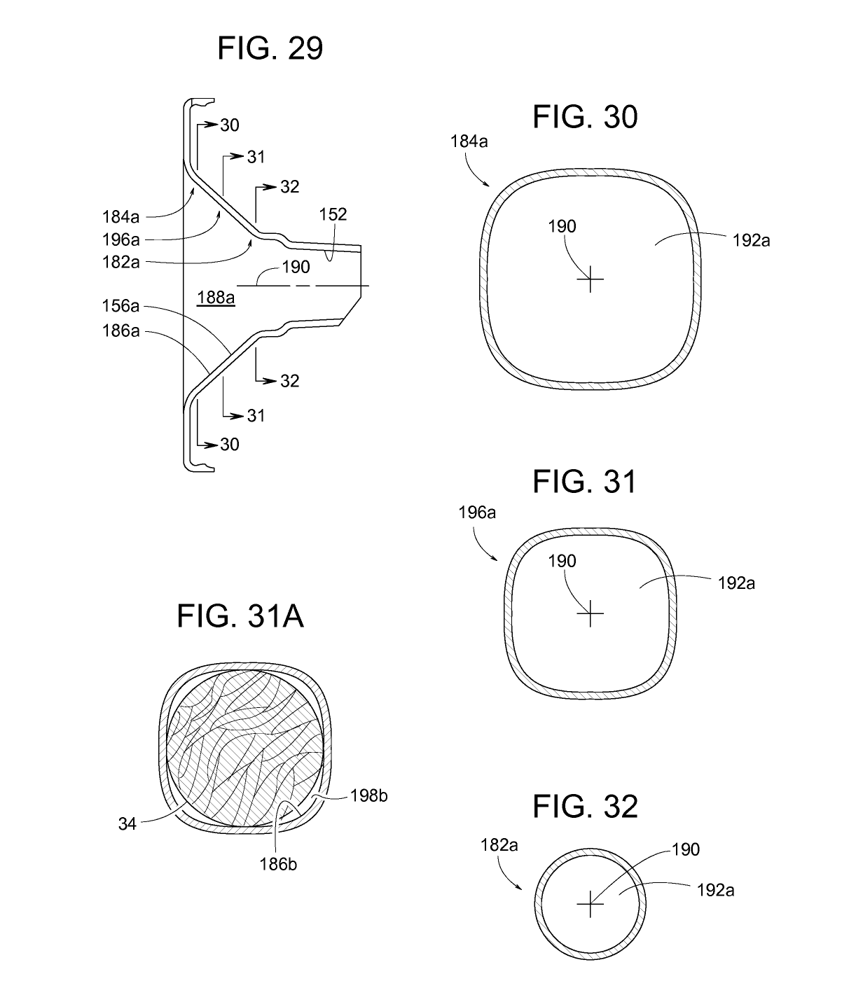

In the example shown in FIGS. 29-32, breast guide 156a comprises a tubular wall 186a converging from a wide end 184a to a narrow end 182a, wherein narrow end 182a adjoins nipple receptacle 152. A tubular wall 186a defines a breast-receiving chamber 188a within breast guide 156a. Breast guide 156a and nipple receptacle 152 define a longitudinal centerline 190 extending centrally through both breast-receiving chamber 188a and nipple receptacle 152. Breast-receiving chamber 188a has a cross-sectional area 192a of varying size lying perpendicular to centerline 190. Cross-sectional area 192a extends radially from centerline 190 to the interior surface of tubular wall 186a and can be taken across any point along centerline 190 between ends 184a and 182a. Cross-sectional area 192a is larger at wide end 184a than at narrow end 182a. At wide end 184a and/or at an intermediate location 196a between ends 184a and 182a, cross-sectional area 192a has a noncircular shape (e.g., superellipse, regular ellipse, rectangle, rounded rectangle, square, rounded square, irregular, polygon, etc.).

In this particular example, the noncircular shape of area 192a (e.g., at wide end 184a and/or at intermediate location 196a) is a superellipse defined by an equation (in the x, y, Cartesian coordinate system) where the sum of a first value and a second value is equal to a total constant (e.g., a total constant equal to one), wherein the first value is the absolute value of a first ratio raised to the nth power, the second value is the absolute value of a second ratio raised to the nth power, the first ratio is the x-coordinate divided by a first constant denominator, the second ratio is the y-coordinate divided by a second constant denominator. In the example shown in FIGS. 30-32, the value of the exponent “n” equals three, and the first constant denominator equals the second constant denominator (a=b). The actual values of the constant denominators and the total constant determine the scale of cross-sectional area 192a at wide end 184a and/or intermediate location 196a. In some examples, the equation is expressed as |x/a|ⁿ+|y/b|ⁿ=1, where “a” is the first constant denominator, “b” is the second constant denominator, “n” is the exponent having a value of “3,” and “1” is the total constant.

In the example illustrated in FIGS. 29-32, the cross-sectional area 192a is a superellipse at both wide end 184a (FIG. 30) and at intermediate location 196a (FIG. 31). From intermediate location 196a, cross-sectional area 192a transitions to being substantially circular at narrow end 182a, as shown in FIG. 32. The circular shape of narrow end 182a provides an effective circumferential seal with breast 34 near nipple 38 while the superelliptical shape at intermediate location 196a provides at least one air vent passageway 198a between breast 34 and tubular wall 194a, as shown in FIG. 31A. Establishing a circumferential seal near nipple 38 at narrow end 182a (e.g., at points 168 and 170 of FIG. 25) in combination with wide end 184a and/or intermediate location 196a being vented enables vacuum to draw nipple 38 straight into nipple receptacle 152.

In some examples where breast guide 156 defines a vent or an air vent passageway, the vent passageway exists within a radial gap (e.g., vent passageway 198a) between the inner surface of a tubular wall (e.g., tubular wall 186a-e) and a circle inscribed within the tubular wall. In FIGS. 31A, 35A, and 39A; breast 34 is a physical example of such a circle.

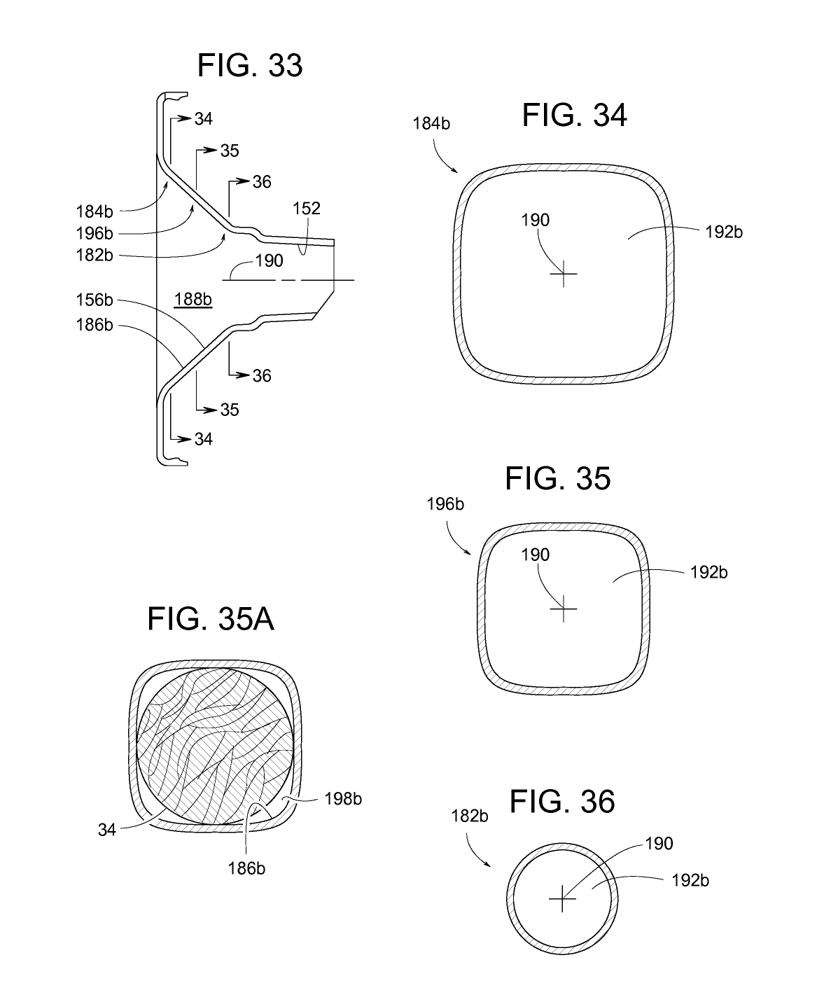

To provide breast guide 156 with a more open vent airway, breast guide 156b is shaped as shown in FIGS. 33-36. In this example, the superelliptical formula applied in FIGS. 34 and 35 has an “n” exponent equal to “4” rather than “3” (FIGS. 30 and 31).

Similar to breast guide 156a of FIGS. 29-32, breast guide 156b of FIGS. 33-36 comprises a tubular wall 186b converging from a wide end 184b to a narrow end 182b, wherein narrow end 182b adjoins nipple receptacle 152. Tubular wall 186b defines a breast-receiving chamber 188b within breast guide 156b. Breast guide 156b and nipple receptacle 152 define longitudinal centerline 190 extending centrally through both breast-receiving chamber 188b and nipple receptacle 152. Breast-receiving chamber 188b has a cross-sectional area 192b of varying size lying perpendicular to centerline 190. Cross-sectional area 192b extends radially from centerline 190 to the interior surface of tubular wall 186b and can be sliced across any point along centerline 190 between ends 184b and 182b. Cross-sectional area 192b is larger at wide end 184b than at narrow end 182b. At wide end 184b and/or at an intermediate location 196b between ends 184b and 182b, cross-sectional area 192b has a noncircular shape.

In this example, the noncircular shape of area 192b (e.g., at wide end 184b and/or at intermediate location 196b) is a superellipse similar to that of FIGS. 30 and 31. In this example, however, the value of the exponent “n” equals four; although, the first constant denominator equals the second constant denominator (a=b). In this case, the equation is expressed as |x/a|⁴+|y/b|⁴=1.

In the example illustrated in FIGS. 33-34, the cross-sectional area 192b is a superellipse at both wide end 184b (FIG. 34) and at intermediate location 196b (FIG. 35). From intermediate location 196b, cross-sectional area 192b transitions to being substantially circular at narrow end 182b, as shown in FIG. 36. The circular shape of narrow end 182b provides an effective circumferential seal with breast 34 near nipple 38 while the superelliptical shape at intermediate location 196b provides at least one air vent passageway 198b between breast 34 and tubular wall 186b, as shown in FIG. 35A. Establishing a circumferential seal near nipple 38 at narrow end 182b (e.g., at points 168 and 170 of FIG. 25) in combination with wide end 184b and/or intermediate location 196b being vented enables vacuum to draw nipple 38 straight into nipple receptacle 152.

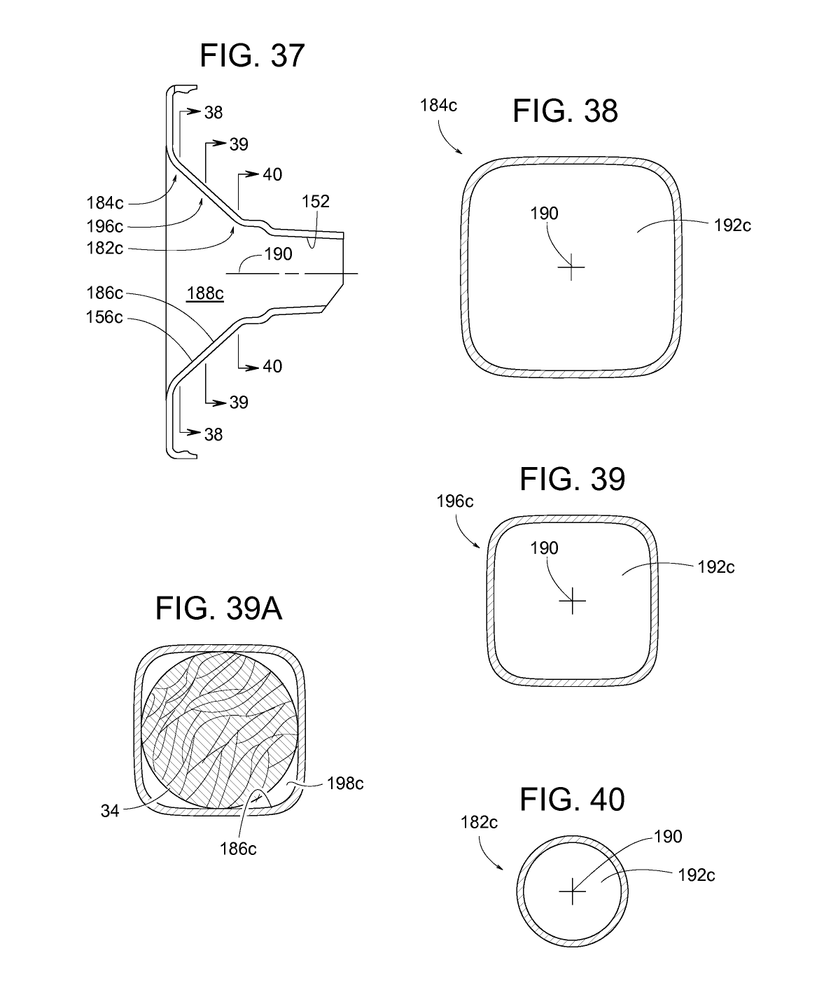

To provide breast guide 156 with an even more open vent airway, breast guide 156c is shaped as shown in FIGS. 37-40. In this example, the superelliptical formula applied in FIGS. 38 and 39 has an “n” exponent equal to “5” rather than “3” (FIG. 30 and 31) or “4” (FIGS. 34 and 35)

Similar to breast guide 156a of FIGS. 29-32, breast guide 156c of FIGS. 37-40 comprises a tubular wall 186c converging from a wide end 184c to a narrow end 182c, wherein narrow end 182c adjoins nipple receptacle 152. Tubular wall 186c defines a breast-receiving chamber 188c within breast guide 156c. Breast guide 156c and nipple receptacle 152 define longitudinal centerline 190 extending centrally through both breast-receiving chamber 188c and nipple receptacle 152. Breast-receiving chamber 188c has a cross-sectional area 192c of varying size lying perpendicular to centerline 190. Cross-sectional area 192c extends radially from centerline 90 to the interior surface of tubular wall 186c and can be taken across any point along centerline 90 between ends 184c and 182c. Cross-sectional area 192c is larger at wide end 184c than at narrow end 182c. At wide end 184c and/or at an intermediate location 196c between ends 184c and 182c, cross-sectional area 192c has a noncircular shape.

In this example, the noncircular shape of area 192c (e.g., at wide end 184c and/or at intermediate location 196c) is a superellipse similar to that of FIGS. 30 and 31. In this example, however, the value of the exponent “n” equals five; although, the first constant denominator equals the second constant denominator (a=b). In this case, the equation is expressed as |x/a|⁵+|y/b|⁵=1.

In the example illustrated in FIGS. 37-40, the cross-sectional area 192c is a superellipse at both wide end 184c (FIG. 38) and at intermediate location 196c (FIG. 39). From intermediate location 196c, cross-sectional area 192c transitions to being substantially circular at narrow end 182c, as shown in FIG. 40. The circular shape of narrow end 182c provides an effective circumferential seal with breast 34 near nipple 38 while the superelliptical shape at intermediate location 196c provides at least one air vent passageway 198c between breast 34 and tubular wall 186c, as shown in FIG. 39A. Establishing a circumferential seal near nipple 38 at narrow end 182c (e.g., at points 168 and 170 of FIG. 25) in combination with wide end 184c and/or intermediate location 196c being vented enables vacuum to draw nipple 38 straight into nipple receptacle 152.

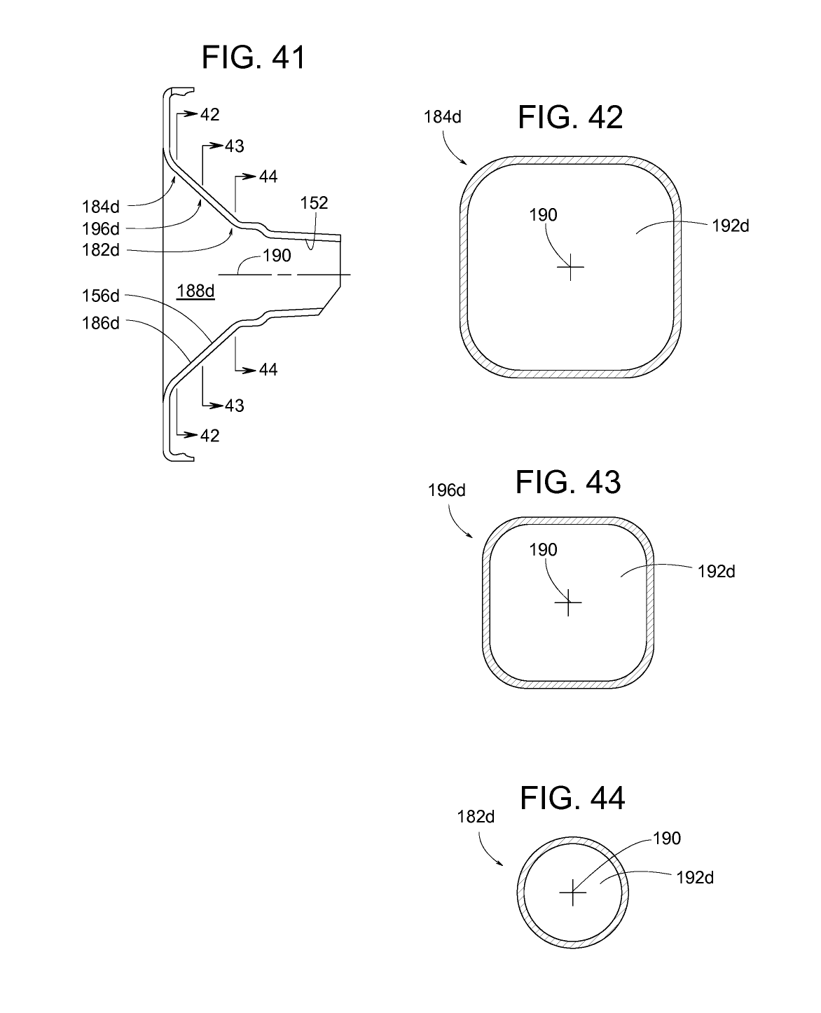

FIGS. 41-44 show an example breast guide 156d having cross-sectional areas that are rounded squares instead of superellipses. Similar to breast guide 156a of FIGS. 29-32, breast guide 156d of FIGS. 41-44 comprises a tubular wall 186d converging from a wide end 184d to a narrow end 182d, wherein narrow end 182d adjoins nipple receptacle 152. Tubular wall 186d defines a breast-receiving chamber 188d within breast guide 156d. Breast guide 156d and nipple receptacle 152 define longitudinal centerline 190 extending centrally through both breast-receiving chamber 188d and nipple receptacle 152. Breast-receiving chamber 188d has a cross-sectional area 192d of varying size lying perpendicular to centerline 190. Cross-sectional area 192d extends radially from centerline 190 to the interior surface of tubular wall 186d and can be taken across any point along centerline 190 between ends 184d and 182d. Cross-sectional area 192d is larger at wide end 184d than at narrow end 182d. At wide end 184d and/or at an intermediate location 196d between ends 189d and 182d, cross-sectional area 192d has a noncircular shape.

In this example, the noncircular shape of area 192d (e.g., at wide end 184d and/or at intermediate location 196d) is a rounded square. In some examples, cross-sectional area 192d is a rounded square at both wide end 184d (FIG. 42) and at intermediate location 196d (FIG. 43). From intermediate location 196d, cross-sectional area 192d transitions to being substantially circular at narrow end 182d, as shown in FIG. 44. The circular shape of narrow end 182d provides an effective circumferential seal with breast 34 near nipple 38 while the rounded square shape at intermediate location 196d provides at least one air vent passageway between breast 34 and tubular wall 186d at the square shape's rounded corners. Establishing a circumferential seal near nipple 38 at narrow end 182d (e.g., at points 168 and 170 of FIG. 25) in combination with wide end 184d and/or intermediate location 196d being vented enables vacuum to draw nipple 38 straight into nipple receptacle 152.

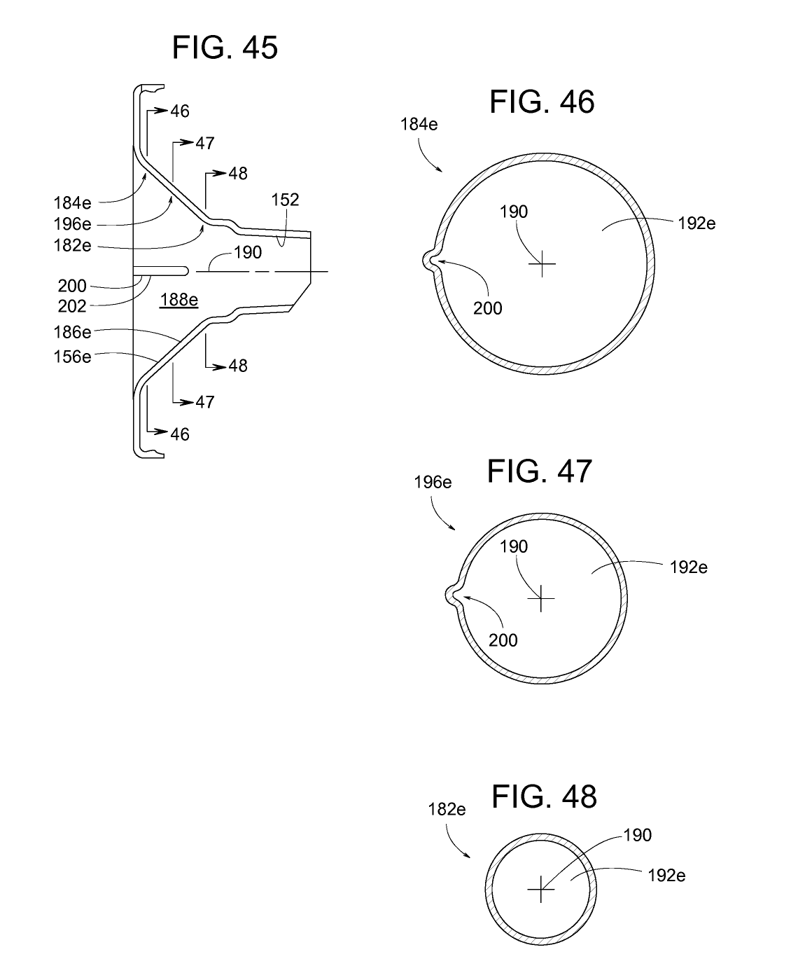

FIGS. 45-48 show an example breast guide 156e having cross-sectional areas that are nearly circular but with the addition of an air vent passageway 200. In some examples, air vent passageway 200 is in the form of a groove 202 that is elongate between a wide end 184e and the narrow end 182e of a tubular wall 186e. Tubular wall 186e defines a breast-receiving chamber 188e within breast guide 156e. Breast guide 156e and nipple receptacle 152 define longitudinal centerline 190 extending centrally through both breast-receiving chamber 188e and nipple receptacle 152. Breast-receiving chamber 188e has a cross-sectional area 192e of varying size lying perpendicular to centerline 190. Cross-sectional area 192e extends radially from centerline 190 to the interior surface of tubular wall 186e and can be taken across any point along centerline 190 between ends 184e and 182e. Cross-sectional area 192e is larger at wide end 184e than at narrow end 182e. At wide end 184e and/or at an intermediate location 196e between ends 184e and 182e, cross-sectional area 192e has a noncircular shape due to the addition of groove 202.

In some examples, cross-sectional area 192e is noncircular (due to groove 202) at both wide end 184e (FIG. 46) and at intermediate location 196e (FIG. 47). From intermediate location 196e, cross-sectional area 192e transitions to being substantially circular at narrow end 182e, as shown in FIG. 48. The circular shape of narrow end 182e provides an effective circumferential seal with breast 34 near nipple 38 while groove 202 at intermediate location 196e provides at least one air vent passageway between breast 34 and tubular wall 186e. Establishing a circumferential seal near nipple 38 at narrow end 182e (e.g., at points 168 and 170 of FIG. 25) in combination with wide end 184e and/or intermediate location 196e being vented enables vacuum to draw nipple 38 straight into nipple receptacle 152.

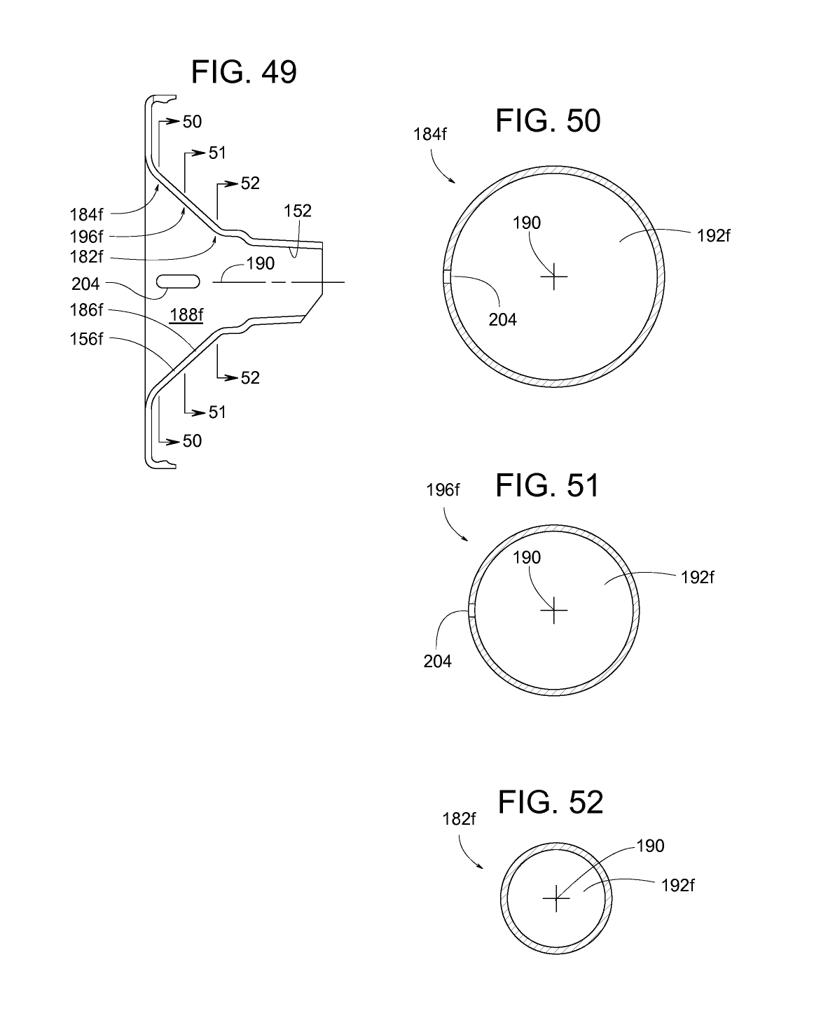

FIGS. 49-52 show an example breast guide 156f having cross-sectional areas that are nearly circular but with the addition of an air vent hole 204 extending radially through a tubular wall 186f of breast guide 156E Vent hole 204 is situated between a wide end 184f and a narrow end 182f of tubular wall 186f. Tubular wall 186f defines a breast-receiving chamber 188f within breast guide 156f. Breast guide 156f and nipple receptacle 152 define longitudinal centerline 190 extending centrally through both breast-receiving chamber 188f and nipple receptacle 152. Breast-receiving chamber 188f has a cross-sectional area 192f of varying size lying perpendicular to centerline 190. Cross-sectional area 192f extends radially from centerline 190 to the interior surface of tubular wall 186f and can be taken across any point along centerline 190 between ends 184f and 182f. Cross-sectional area 192f is larger at wide end 184f than at narrow end 182f. At wide end 184f and/or at an intermediate location 196f between ends 184f and 182f, cross-sectional area 192f is open through air vent hole 204.

In some examples, cross-sectional area 192f is noncircular (due to hole 204) at both wide end 184f (FIG. 50) and at intermediate location 196f (FIG. 51). From intermediate location 196f, cross-sectional area 192f transitions to being substantially circular at narrow end 182f, as shown in FIG. 52. The circular shape of narrow end 182f provides an effective circumferential seal with breast 34 near nipple 38 while air vent hole 204 at intermediate location 196f provides at least one air vent passageway at breast 34 and tubular wall 186f. Establishing a circumferential seal near nipple 38 at narrow end 182f (e.g., at points 168 and 170 of FIG. 25) in combination with wide end 184f and/or intermediate location 196f being vented enables vacuum to draw nipple 38 straight into nipple receptacle 152.

For further clarification, the term, “suction tube” refers to any conduit having a tubular wall of sufficient thickness, stiffness, and/or strength to convey air at subatmospheric pressure. In some examples, suction tube 48 is more flexible than outer shell 24, breast receiver 22, and/or fluid exchanger 26. Such tube flexibility makes tube 48 easier to use and fit to fluid exchanger 26. The term, “coupled to” refers to two members being connected either directly without an intermediate connecting piece or being connected indirectly via an intermediate connecting piece between the two members. The term, “coupled to” encompasses permanent connections (e.g., bonded, welded, etc.), seamless connections (e.g., the two members are of a unitary piece), and separable connections. The term, “opening” of a fluid pathway refers to a cross-sectional area through which fluid is directed to flow in a direction generally perpendicular to the area as guided by the fluid pathway. The term, “radial gap” refers to clearance as measured in a direction perpendicular to longitudinal centerline 58. The terms, “negative pressure,” “subatmospheric pressure,” and “vacuum” all refer to a pressure that is less than atmospheric pressure. The term, “positive pressure,” refers to a pressure that is greater than atmospheric pressure. Storage chamber 42 is not necessarily for long term storage but rather for collecting and temporarily storing milk 14 as the lactating woman is expressing milk. In some examples, milk collection device 12 includes a slot-and-key 144 alignment feature (FIG. 8) that establishes a certain desired rotational alignment (about longitudinal centerline 58) between fluid exchanger 26 and breast receiver 22. The term, “extending centrally” as it pertains to a centerline extending through a chamber and a receptacle means that when a first circle is inscribed within the chamber and a second circle is inscribed within the receptacle, the center points of the first and second circles lie on the centerline, and the centerline is perpendicular to the planes of both circles. A rounded rectangle is a rectangle with four straight sides and curved corners. The term, “circular” as it pertains to an area means that the area's perimeter is a continuous 360-degree circle and not just part of circle. The term, “superelliptical” refers to an area having the shape of a superellipse. A superellipse is one example of a regular ellipse. In some examples, the funnel-shaped, breast-receiving breast guides disclosed herein are used in breast milk collection devices that are not necessarily worn within the cup of a special-purpose or ordinary brassier. In some examples, transitioning or blending a funnel-shaped breast guide from a superellipse at one location to a circle at the narrow end of the breast guide is accomplished by gradually reducing the exponent “n” of the superellipse to a value of “2” at the narrow end. In some examples, the funnel-shaped, breast-receiving breast guides disclosed herein are adapted for use with FREEMIE style breast pump systems, wherein FREEMIE is a registered trademark of DAO Health of Sacramento, Calif. In some examples, the funnel-shaped, breast-receiving breast guides disclosed herein are adapted for use with MEDELA style breast pump systems, wherein MEDELA is a registered trademark of Medela Holding AG of Barr, Switzerland.

Although the invention is described with respect to a preferred embodiment, modifications thereto will be apparent to those of ordinary skill in the art. The scope of the invention, therefore, is to be determined by reference to the following claims:

1. A breast pump system usable by a lactating woman for collecting milk, the breast pump system comprising:
a nipple receptacle adapted to receive a nipple of the lactating woman; and
a breast guide being adapted to receive a breast of the lactating woman, the breast guide having a tubular wall converging from a wide end to a narrow end of the breast guide, the narrow end of the breast guide adjoining the nipple receptacle, the tubular wall defining a breast-receiving chamber, the breast guide and the nipple receptacle defining a longitudinal centerline extending centrally through both the breast-receiving chamber and the nipple receptacle, the breast-receiving chamber having a cross-sectional area of varying size lying perpendicular to the longitudinal centerline, the cross-sectional area extending radially from the longitudinal centerline to the tubular wall, the cross-sectional area being larger at the wide end than at the narrow end, the cross-sectional area having a noncircular shape at an intermediate location between the wide end and the narrow end.

2. The breast pump system of claim 1, wherein the cross-sectional area of the breast chamber is substantially elliptical at the intermediate location between the wide end and the narrow end of the tubular wall.

3. The breast pump system of claim 1, wherein the cross-sectional area of the breast chamber is substantially superelliptical at the intermediate location between the wide end and the narrow end of the tubular wall.

4. The breast pump system of claim 1, wherein the cross-sectional area of the breast chamber is a rounded rectangle at the intermediate location between the wide end and the narrow end of the tubular wall.

5. The breast pump system of claim 1, wherein the cross-sectional area of the breast chamber is a rounded square at the intermediate location between the wide end and the narrow end of the tubular wall.

6. The breast pump system of claim 1, wherein the tubular wall defines an air vent passageway in the form of a groove that is elongate between the wide end and the narrow end of the tubular wall.

7. The breast pump system of claim 6, wherein the groove is in direct open fluid communication with breast chamber.

8. The breast pump system of claim 1, wherein the tubular wall defines an air vent hole extending radially through the tubular wall between the wide end and the narrow end of the tubular wall.

9. The breast pump system of claim 1, wherein the cross-sectional area of the breast chamber is noncircular at the wide end.

10. The breast pump system of claim 1, wherein the cross-sectional area of the breast chamber is substantially circular at the narrow end.

11. A breast pump system usable by a lactating woman for collecting milk, the breast pump system comprising:
a nipple receptacle adapted to receive a nipple of the lactating woman; and
a breast guide being adapted to receive a breast of the lactating woman, the breast guide having a tubular wall converging from a wide end to a narrow end of the breast guide, the narrow end of the breast guide adjoining the nipple receptacle, the tubular wall defining a breast-receiving chamber, the breast guide and the nipple receptacle defining a longitudinal centerline extending centrally through both the breast-receiving chamber and the nipple receptacle, the breast-receiving chamber having a cross-sectional area of varying size lying perpendicular to the longitudinal centerline, the cross-sectional area extending radially from the longitudinal centerline to the tubular wall, the cross-sectional area being larger at the wide end than at the narrow end, the cross-sectional area having an intermediate location between the wide end and the narrow end, the cross-sectional area having a greater deviation from a circle at the intermediate location than at the narrow end.

12. The breast pump system of claim 11, wherein the cross-sectional area of the breast chamber is substantially superelliptical at the intermediate location between the wide end and the narrow end of the tubular wall.

13. The breast pump system of claim 11, wherein the tubular wall defines an air vent passageway in the form of a groove that is elongate between the wide end and the narrow end of the tubular wall.

14. The breast pump system of claim 13, wherein the groove is in direct open fluid communication with breast chamber.

15. The breast pump system of claim 11, wherein the tubular wall defines an air vent hole extending radially through the tubular wall between the wide end and the narrow end of the tubular wall.

16. The breast pump system of claim 11, wherein the cross-sectional area of the breast chamber is noncircular at the wide end.

17. A breast pump system usable by a lactating woman for collecting milk, the breast pump system comprising:
a nipple receptacle adapted to receive a nipple of the lactating woman; and
a breast guide being adapted to receive a breast of the lactating woman, the breast guide having a tubular wall converging from a wide end to a narrow end of the breast guide, the narrow end of the breast guide adjoining the nipple receptacle, the tubular wall defining a breast-receiving chamber, the breast guide and the nipple receptacle defining a longitudinal centerline extending centrally through both the breast-receiving chamber and the nipple receptacle, the tubular wall defining an air vent passageway lying against the tubular wall and extending along an elongate path between the wide end and the narrow end of the tubular wall.

18. The breast pump system of claim 17, wherein the elongate path is angularly displaced out of parallel alignment with the longitudinal centerline.

19. The breast pump system of claim 17, wherein the breast-receiving chamber has a cross-sectional area of varying size lying perpendicular to the longitudinal centerline, the cross-sectional area extending radially from the longitudinal centerline to the tubular wall, the cross-sectional area being larger at the wide end than at the narrow end, the cross-sectional area having a noncircular shape at an intermediate location between the wide end and the narrow end.

20. The breast pump system of claim 19, wherein the air vent passageway is in direct open fluid communication with breast chamber.
